(19) United States
(12) Patent Application Publication
(10) Pub. No.: US 2020/0086133 A1
(43) Pub. Date: Mar. 19, 2020

(54) VALIDATION, COMPLIANCE, AND/OR INTERVENTION WITH EAR DEVICE

(71) Applicant: BIOINTELLISENSE, INC., Redwood City, CA (US)

(72) Inventors: David Jonq WANG, Palo Alto, CA (US); James R. MAULT, Evergreen, CO (US); Brian Chris RO, Redwood City, CA (US); Henry WeiKang LEUNG, Sunnyvale, CA (US)

(21) Appl. No.: 16/574,781

(22) Filed: Sep. 18, 2019

Related U.S. Application Data
(60) Provisional application No. 62/732,922, filed on Sep. 18, 2018.

(51) Int. Cl. A61N 2/00; A61B 5/0205; A61B 5/01; A61B 5/0476; A61B 5/00; A61B 5/11; A61B 5/0402; A61B 5/16; A61N 1/36; G10L 15/22; H04R 1/10

(52) U.S. Cl. CPC A61N 2/004; G06N 20/00; A61B 5/01; A61B 5/0476; A61B 5/4557; A61B 5/4806; A61B 5/4845; A61B 5/1118; A61B 5/0402; A61B 5/6803; A61B 5/746; A61B 5/7405; A61B 5/749; A61B 5/486; A61B 5/165; A61B 5/747; A61N 1/36036; A61B 5/1117; A61B 5/0006; A61B 5/0008; A61B 5/0022; G10L 15/22; H04R 1/10; A61B 5/02055

(57) ABSTRACT

Some embodiments relate to ear-mountable devices with one or more sensors and both input and output capabilities. Such ear-mountable devices may validate behaviors, biometrics, and/or environmental conditions by generating a signal indicative of the same at an ear of the user and then determining the behaviors, biometrics, and/or environmental conditions based on the signal. Such ear-mountable devices may determine compliance of a user by outputting, through an audio output device of the ear-mountable devices, a compliance message to evoke a target behavior in the user, monitoring behavior of the user through a sensor of the ear-mountable device, and determining compliance of the user with the target behavior based on the monitoring. Such ear-mountable devices may implement intervention by determining a state of a user, determining whether the state warrants an intervention or treatment, determining a specific intervention or treatment to administer when warranted, and administering the specific intervention or treatment.

### CROSS-REFERENCE TO RELATED APPLICATION

This application claims the benefit of and priority to U.S. Provisional Application No. 62/732,922, filed Sep. 18, 2018, which is incorporated herein by reference. This application is related to co-pending U.S. application Ser. No. 16/118,242 (hereinafter the '242 application), filed Aug. 30, 2018. The '242 application is incorporated herein by reference.

### FIELD

Some embodiments described herein generally relate to validation, compliance, and/or intervention with an ear device.

### BACKGROUND

Unless otherwise indicated herein, the materials described herein are not prior art to the claims in the present application and are not admitted to be prior art by inclusion in this section.

Sound-related behaviors such as sneezing, coughing, vomiting, and/or shouting (e.g., tied to mood or rage) may be useful to measure in health-related research. For example, measuring sneezing, coughing, vomiting, and/or shouting may be useful in researching the intended effects and/or side effects of a given medication. Such behaviors have been self-reported in the past, but self-reporting may be cumbersome to subjects, may be inefficient, and/or may be inaccurate.

The subject matter claimed herein is not limited to implementations that solve any disadvantages or that operate only in environments such as those described above. Rather, this background is only provided to illustrate one example technology area where some implementations described herein may be practiced.

### SUMMARY

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

Some example implementations described herein generally relate to validation, compliance, and/or intervention with an ear device.

An example validation method may include generating, at an ear of a user, a signal indicative of at least one of a behavior of the user, a biometric of the user, or an environmental condition of an environment of the user. The method may also include determining, based on the signal, at least one of the behavior of the user, the biometric of the user, or the environmental condition of the environment of the user.

An example compliance method may include outputting, through an audio output device positioned at least partially in, on, or proximate to an ear of a user, a compliance message to evoke a target behavior in the user. The method may also include monitoring behavior of the user, through a sensor positioned in, on, or proximate to the ear of the user. The method may also include determining, based on the monitoring, compliance of the user with the target behavior.

An example intervention method may include determining a state of a user. The method may include determining whether the state of the user warrants an intervention or treatment. The method may include in response to determining that the state of the user warrants an intervention or treatment, determining a specific intervention or treatment to administer to the user. The method may include administering the specific intervention or treatment to the user. The state of the user may be determined based on a signal generated by a sensor positioned in, on, or proximate to the user's ear and/or the specific intervention or treatment may be administered at least in part by an output device positioned in, on, or proximate to the user's ear.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates an example operating environment;

FIG. 2A is a block diagram of an ear-mountable device and remote server of FIG. 1;

FIGS. 2B and 2C illustrate two ear-mountable devices implemented as hearing aids;

FIG. 2D illustrates an ear-mountable device implemented as circumaural headphones;

FIG. 3 is a flowchart of an example validation method;

FIG. 4 is a flowchart of an example compliance method;

FIG. 5 is a flowchart of an example intervention method,

all arranged in accordance with at least one embodiment described herein.

### DETAILED DESCRIPTION OF SOME EXAMPLE EMBODIMENTS

Some embodiments described herein generally relate to validation, compliance, and/or intervention with an ear device, such as a hearing aid or headphone. The '242 application discloses methods, systems, and/or devices related to sensor fusion to validate and/or measure sound-producing behaviors of a subject. Such sound-producing behaviors can include sneezing, coughing, vomiting, shouting, or other sound-producing behaviors. The embodiments described in the '242 application may detect sound-producing behaviors in general and/or may categorize each of the sound-producing behaviors, e.g., as a sneeze, cough, vomiting, wheezing, shortness of breath, chewing, swallowing, masturbation, sex, a shout, or other particular type of sound-producing behavior.

Sensors implemented in the '242 application may be included in a wearable electronic device worn on a user's wrist, included in a user's smartphone (often carried in a user's pocket), or applied to a user's body, e.g., in the form of a sensor sticker. Such devices are often at least partially covered by a user's clothing some or all of the time during use. The presence of clothing may interfere with sensor detection, introducing noise and/or otherwise reducing measurement accuracy.

In comparison, hearing aids, headphones, and other ear-mountable devices may be less likely to be even partially covered by clothing than wrist-wearable devices, smartphones, sensor stickers, and/or other wearable electronic devices. For example, many users when clothed keep their heads completely uncovered such that any ear-mountable device worn by the user may remain uncovered. Further, many head-wearable accessories, such as baseball caps and bandanas, may interfere little or not at all with an ear-mountable device.

Some embodiments described herein relate to ear-mountable devices with one or more sensors and both input and output capabilities. Ear-mountable devices may be advantageously mounted (e.g., worn on or otherwise attached to) to a user's ears on the user's head where it is unlikely to be covered by clothing or other objects that may interfere with sensing functions of the devices. In addition, ear-mountable devices may include one or more sensors in contact with or proximate to the user's ear canal, which may have solid vibration and sound conduction through the user's skull, such that the ear-mountable devices may sense solid vibrations and/or sounds from the user's ear canal. Further, the proximity to the user's head may permit ear-mountable devices to sense brain waves and/or electroencephalography (EEG) waves.

Due to the location of ear-mountable devices when used, e.g., on the user's head, they may be better situated than other personal wearable electronic devices to detect with less noise and/or better accuracy one or more of the following parameters: core body temperature, ambient light exposure, ambient ultraviolet (UV) light exposure, ambient temperature, head orientation, head impact, coughing, sneezing, and/or vomiting.

In some embodiments, an ear-mountable device may include an output device, such as a speaker, that outputs information in an audio format to be heard by a user. Alternatively or additionally, an ear-mountable device may include an input device, such as a microphone or an accelerometer, through which a user may provide input. Accordingly, embodiments described herein may use an ear-mountable device for: passive and/or active validation of a behavior, an environmental condition, and/or a biometric of the use; compliance; and/or intervention.

Each ear-mountable device may be implemented as a hearing aid, a headphone, or other device configured to be mounted to a user's ear. Hearing aid users often wear and use their hearing aids for lengths of time that may be longer than lengths of times for which headphones may typically be used. Even so, embodiments described herein may be implemented in either or both hearing aids and headphones, or in other ear-mountable devices, with or without regard to an expected or typical period of use of such devices.

Reference will now be made to the drawings to describe various aspects of some example embodiments of the disclosure. The drawings are diagrammatic and schematic representations of such example embodiments, and are not limiting of the present disclosure, nor are they necessarily drawn to scale.

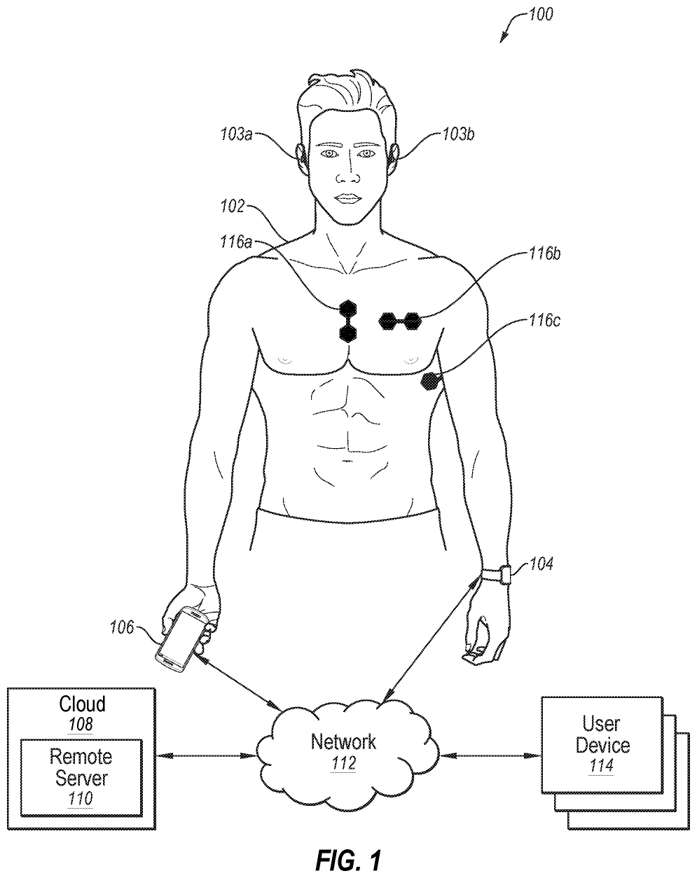

FIG. 1 illustrates an example operating environment 100 (hereinafter “environment 100”), arranged in accordance with at least one embodiment described herein. The environment 100 includes a subject 102 and one or more ear-wearable electronic devices 103a, 103b (hereinafter generally “ear-mountable device 103” or “ear-mountable devices 103”). The environment 100 may additionally include a wearable electronic device 104, a smartphone 106 (or other personal electronic device), a cloud computing environment (hereinafter “cloud 108”) that includes at least one remote server 110, a network 112, multiple third party user devices 114 (hereinafter “user device 114” or “user devices 114”), and multiple third parties (not shown). The user devices 114 may include wearable electronic devices and/or smartphones of other subjects or users not illustrated in FIG. 1. The environment 100 may additionally include one or more sensor devices 116, such as the devices 116a, 116b, and/or 116c, implemented as sensor stickers that attach directly to skin of the user 102.

The network 112 may include one or more wide area networks (WANs) and/or local area networks (LANs) that enable the ear-mountable devices 103, the wearable electronic device 104, the smartphone 106, the cloud 108, the remote server 110, the sensor devices 116, and/or the user devices 104 to communicate with each other. In some embodiments, the network 112 includes the Internet, including a global internetwork formed by logical and physical connections between multiple WANs and/or LANs. Alternately or additionally, the network 112 may include one or more cellular RF networks and/or one or more wired and/or wireless networks such as 802.xx networks, Bluetooth access points, wireless access points, IP-based networks, or other suitable networks. The network 112 may also include servers that enable one type of network to interface with another type of network.

One or more of the ear-mountable device 103, the wearable electronic device 104, the smartphone 106, and/or the sensor devices 116 may include a sensor configured to generate data signals that measure parameters that may be indicative of behaviors, environmental conditions, and/or biometric responses of the subject 102. The measured parameters may include, for example, sound near the subject 102, acceleration of the subject 102 or of a head, chest, hand, wrist, or other part of the subject 102, angular velocity of the subject 102 or of a head, chest, hand, wrist, or other part of the subject 102, temperature of the skin of the subject 102, core body temperature of the subject 102, blood oxygenation of the subject 102, blood flow of the subject 102, electrical activity of the heart of the subject 102, electrodermal activity (EDA) of the subject 102, sound or vibration or other parameter indicative of the subject 102 swallowing, grinding teeth, or chewing, an intoxication state of the subject 102, a dizziness level of the subject 102, EEG brain waves of the subject 102, one or more parameters indicative of volatile organic compounds in the user's sweat or sweat vapor, an environmental or ambient temperature, light level, or UV light level of an environment of the user, or other parameters, one or more of which may be indicative of certain sound-producing behaviors of the subject 102, such as sneezing, coughing, wheezing, vomiting, or shouting. The ear-mountable device 103, the wearable electronic device 104, the smartphone 106, and/or the remote server 110 may be configured to determine or extract one or more features from the data signals and/or from data derived therefrom to validate behaviors, environmental conditions, or biometrics of the user and or to implement compliance and/or interventions for the subject 102.

In some embodiments, one or both of the ear-mountable devices 103 may include a sensor and/or input device that may be positioned at any desired location in, on, or proximate to the ear. Example locations for each sensor and/or input device of each of the ear-mountable devices 103 include in the user's ear canal, in or near the user's tympanic membrane, in the user's ear-hole (e.g., the opening of the ear canal), behind the user's ear, on the user's ear lobe, or other suitable location(s) in, on, or proximate to the user's ear. For example, a sensor to acquire core body temperature, heart rate via photoplethysmograph (PPG), sweat vapor, signals relating to the tympanic membrane, and/or UV/light levels may be positioned inside the user's ear canal. Alternatively or additionally, a sensor to acquire environmental/ambient temperature/light levels/sound may be positioned behind the user's ear.

All of the sensors may be included in a single device, such as the ear-mountable device 103, the sensor device 116, the wearable electronic device 104, and/or the smartphone 106. Alternately or additionally, the sensors may be distributed between two or more devices. For instance, one or each of the ear-mountable device 103, the sensor devices 116, the wearable electronic device 104 or the smartphone 106 may include a sensor. Alternately or additionally, the one or more sensors may be provided as separate sensors that are separate from either of the ear-mountable device 103, the wearable electronic device 104, or the smartphone 106. For example, the sensor devices 116 may be provided as separate sensors. In particular, the sensor devices 116 are separate from the ear-mountable device 103, the wearable electronic device 104, and the smartphone 106.

Each sensor, such as each sensor included in the ear-mountable device 103, may include any of a discrete microphone, an accelerometer, a gyro sensor, a thermometer, an oxygen saturation sensor, a PPG sensor, an electrocardiogram (ECG) sensor, an EDA sensor, or other sensor. In some embodiments, each of the ear-mountable devices 103 may include multiple sensors. Alternatively or additionally, a first sensor device 116a may be positioned along a sternum of the subject 102, a second sensor device 116b may be positioned over the left breast to be over the heart, and/or a third sensor device 116c may be positioned beneath the left arm of the subject 102. In these and other embodiments, the different sensors included in, e.g., two or more of the ear-mountable device 103, the wearable electronic device 104, the smartphone 106, and/or the sensor devices 116 at different locations may be beneficial for a more robust set of data to analyze the subject 102. For example, different locations of the sensors may identify different features based on their respective locations proximate different parts of the anatomy of the subject 102.

In some embodiments, the sensor(s) included in one or more of the ear-mountable device 103, the wearable electronic device 104, the smartphone 106, and/or the sensor devices 116 may include a discrete or integrated sensor attached to or otherwise borne on the body of the subject 102. Various non-limiting examples of sensors that may be attached to the body of the subject 102 or otherwise implemented according to the embodiments described herein and that may be implemented as the sensor(s) included in the ear-mountable device 103, the wearable electronic device 104, the smartphone 106, and/or the sensor devices 116 include microphones, PPG sensors, accelerometers, gyro sensors, heart rate sensors (e.g., pulse oximeters), ECG sensors, EDA sensors, or other suitable sensors. Each sensor may be configured to generate data signals, e.g., of sounds, vibrations, acceleration, angular velocity, blood flow, electrical activity of the heart, EDA, temperature, light level, UV light level, or of other parameters of or near the subject 102.

In an example implementation, at least one ear-mountable device 103 is provided with at least one sensor in the form of a microphone. Alternatively or additionally the ear-mountable device 103 may include an output device such as a speaker which may be used both for a normal output function of a hearing aid (e.g., to amplify sounds for a user) or headphone (e.g., as audio output from a music player or other device) as well as to output messages to a user for active validation, compliance, and/or intervention.

Each of the ear-mountable devices 103, the wearable electronic device 104, and/or the sensor devices 116 may be embodied as a portable electronic device and may be borne by the subject 102 throughout the day and/or at other times. As used herein, “borne by” means carried by and/or attached to. One or more of the ear-mountable devices 103, the wearable electronic device 104, the smartphone 106, and/or the sensor devices 116 may be configured to, among other things, analyze signals collected by one or more sensors within the environment 100 to validate behaviors and/or to implement compliance and/or interventions. Each of the ear-mountable devices 103, the wearable electronic device 104, the smartphone 106, and/or the sensor devices 116 may analyze and process sensor signals individually, or one or more of the ear-mountable devices 103, the wearable electronic device 104, the smartphone 106, and/or the sensor devices 116 may collect sensor signals from some or all of the other devices to analyze and/or process multiple sensor signals.

The ear-mountable devices 103, the wearable electronic device 104, the smartphone 106, and/or the sensor devices 116 may be used by the subject 102 to perform journaling, including providing subjective annotations to confirm or deny the occurrence of one or more behaviors, biometrics, and/or environmental conditions. Additional details regarding example implementations of journaling using a wearable electronic device or other device are disclosed in U.S. Pat. No. 10,362,002 issued on Jul. 23, 2019, which is incorporated herein by reference. The subject 102 may provide annotations any time desired by the subject 102, such as after exhibiting a behavior or biometric or after occurrence of an environmental condition and without being prompted by any of the ear-mountable devices 103, the wearable electronic device 104, the smartphone 106, or the sensor devices 116. Alternatively or additionally, the subject 102 may provide annotations regarding a behavior, biometric, or environmental condition responsive to prompts from any of the ear-mountable devices 103, the wearable electronic device 104, the smartphone 106, and/or the sensor devices 116. For instance, in response to detecting a behavior based on data signals generated by one or more sensors, one of the ear-mountable devices 103 or the wearable electronic device 104 may provide an output to the subject 102 to query whether the detected behavior actually occurred. The subject 102 may then provide an annotation or other input that confirms or denies occurrence of the detected behavior. The annotations may be provided to the cloud 108 and in particular to the remote server 110.

The remote server 110 may include a collection of computing resources available in the cloud 108. The remote server 110 may be configured to receive annotations and/or data derived from data signals collected by one or more sensors or other devices, such as the era-mountable devices 103, the wearable electronic device 104, the smartphone 106, and/or the sensor devices 116 within the environment 100. Alternatively or additionally, the remote server 110 may be configured to receive from the sensors relatively small portions of the data signals, or even larger portions or all of the data signals. The remote server 110 may apply processing to the data signals, portions thereof, or data derived from the data signals and sent to the remote server 110, to extract features and/or determine behaviors, biometrics, and/or environmental conditions of the subject 102.

In some embodiments, one or more of the ear-mountable devices 10, the wearable electronic device 104, the smartphone 106, and/or the sensor devices 116 may transmit the data signals to the remote server 110 such that the remote server 110 may detect the behavior, biometric, and/or environmental condition. Additionally or alternatively, one or more of the ear-mountable devices 10, the wearable electronic device 104, the smartphone 106, and/or the sensor devices 116 may detect the behavior, biometric, and/or environmental condition from the data signals locally at one or more of the ear-mountable devices 10, the wearable electronic device 104, the smartphone 106, and/or the sensor devices 116.

In these and other embodiments, a determination of whether or not to perform the detection of the behavior, biometric, and/or environmental condition locally or remotely may be based on capabilities of the processor of the local device, power capabilities of the local device, remaining power of the local device, communication channels available to transmit data to the remote server 110 (e.g., Wi-Fi, Bluetooth, etc.), payload size (e.g., how much data is being communicated), cost for transmitting data (e.g., a cellular connection vs. a Wi-Fi connection), or other criteria. For example, if the ear-mountable device 103 includes a battery as a power source that is not rechargeable, the ear-mountable device 103 may include simple behavior, biometric, or environmental condition detection, and otherwise may send the data signals to the remote server 110 for processing. As another example, if the ear-mountable device 103 includes a rechargeable battery that is full, the ear-mountable device 103 may perform the detection locally when the battery is full or close to full and may decide to perform the detection remotely when the battery has less charge.

As described in the present disclosure, the detection of the behavior, biometric, and/or environmental condition may include one or more steps, such as feature extraction, identification, and/or classification. In these and other embodiments, any of these steps or processes may be performed at any combination of devices such as at the ear-mountable device 103, the wearable electronic device 104, the smartphone 106, the sensor device 116, and/or the remote server 110. For example, the ear-mountable device 103 may collect data and perform some processing on the data (e.g., collecting audio data and performing a power spectral density process on the data), provide the processed data to the smartphone 106, and the smartphone 106 may extract one or more features in the processed data, and may communicate the extracted features to the remote server 110 to classify the features into one or more behaviors.

In some embodiments, an intermediate device may act as a hub to collect data from the ear-mountable device 103, the wearable electronic device 104, the smartphone 106, and/or the sensor device 116. For example, the hub may collect data over a local communication scheme (Wi-Fi, Bluetooth, near-field communications (NFC), etc.) and may transmit the data to the remote server 110. In some embodiments, the hub may act to collect the data and periodically provide the data to the remote server 110, such as once per week. An example hub and associated methods and devices are disclosed in U.S. application Ser. No. 16/395,052 filed Apr. 25, 2019, which is incorporated herein by reference.

The remote server 110 may maintain one or more of the algorithms and/or state machines used in the detection of behaviors, biometrics, and/or environmental conditions by the ear-mountable device 103, the wearable electronic device 104, the smartphone 106, and/or the sensor device 116. In some embodiments, annotations or other information collected by, e.g., the ear-mountable device 103, the wearable electronic device 104, the smartphone 106, the sensor device 116, and/or the user devices 114, for multiple subjects may be fed back to the cloud 108 to update the algorithms and/or state machines. This may lead to significant network effects, e.g., as more information is collected from more subjects, the algorithms and/or state machines used to detect behaviors, biometrics, and/or environmental conditions may be updated to become increasingly accurate and/or efficient. The updated algorithms and/or state machines may be downloaded from the remote server 110 to the ear-mountable device 103, the wearable electronic device 104, the smartphone 106, the sensor device 116, and/or the user devices 114 to, e.g., improve detection.

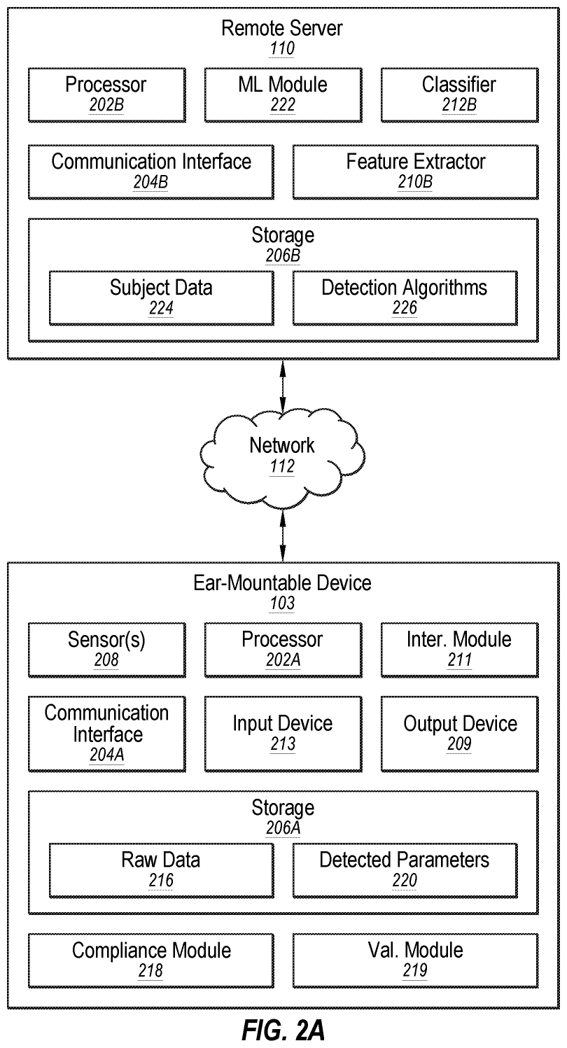

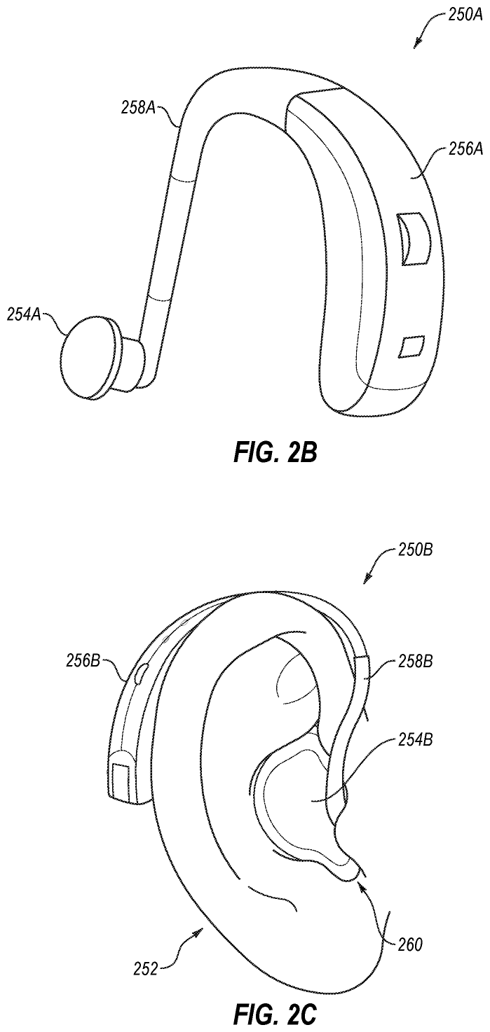

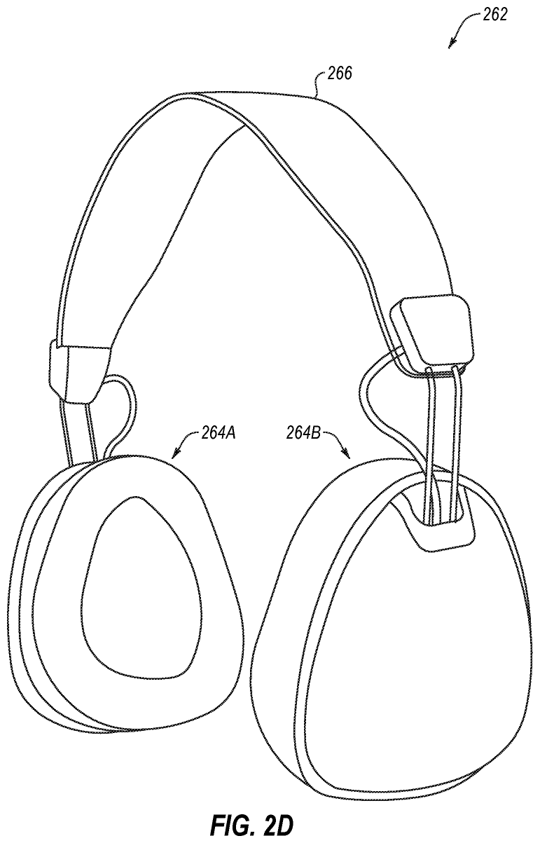

FIG. 2A is a block diagram of the ear-mountable device 103 and remote server 110 of FIG. 1, arranged in accordance with at least one embodiment described herein. Each of the ear-mountable device 103 and the remote server 110 may include a processor 202A or 202B (generically “processor 202”, collectively “processors 202”), a communication interface 204A or 204B (generically “communication interface 204”, collectively “communication interfaces 204”), and a storage and/or memory 206A or 206B (generically and/or collectively “storage 206”). Although not illustrated in FIG. 2A, the wearable electronic device 104, the smartphone 106 (or other personal electronic device), and/or one or more of the sensor devices 116 of FIG. 1 may be configured in a similar or analogous manner as the ear-mountable device 103 as illustrated in FIG. 2A. For instance, the wearable electronic device 104 may include the same, similar, and/or analogous elements or components as illustrated for the ear-mountable device 103 of FIG. 2A.

Each of the processors 202 may include an arithmetic logic unit, a microprocessor, a general-purpose controller, or some other processor or array of processors, to perform or control performance of operations as described herein. The processors 202 may be configured to process data signals and may include various computing architectures including a complex instruction set computer (CISC) architecture, a reduced instruction set computer (RISC) architecture, or an architecture implementing a combination of instruction sets. Although each of the ear-mountable device 103 and the remote server 110 of FIG. 2A includes a single processor 202, multiple processor devices may be included and other processors and physical configurations may be possible. The processor 202 may be configured to process any suitable number format including two's compliment numbers, integers, fixed binary point numbers, and/or floating point numbers, etc. all of which may be signed or unsigned.

Each of the communication interfaces 204 may be configured to transmit and receive data to and from other devices and/or servers through a network bus, such as an I²C serial computer bus, a universal asynchronous receiver/transmitter (UART) based bus, or any other suitable bus. In some implementations, each of the communication interfaces 204 may include a wireless transceiver for exchanging data with other devices or other communication channels using one or more wireless communication methods, including IEEE 802.11, IEEE 802.16, BLUETOOTH®, Wi-Fi, Zigbee, near field communication (NFC), or another suitable wireless communication method.

The storage 206 may include a non-transitory storage medium that stores instructions or data that may be executed or operated on by a corresponding one of the processors 202. The instructions or data may include programming code that may be executed by a corresponding one of the processors 202 to perform or control performance of the operations described herein. The storage 206 may include a non-volatile memory or similar permanent storage media including a flash memory device, an electrically erasable and programmable read only memory (EEPROM), a magnetic memory device, an optical memory device, or some other mass storage for storing information on a more permanent basis. In some embodiments, the storage 206 may also include volatile memory, such as a dynamic random access memory (DRAM) device, a static random access memory (SRAM) device, or other suitable volatile memory device.

The ear-mountable device 103 may additionally include one or more sensors 208, an output device 209, an intervention module 211 (“Inter. Module 211” in FIG. 2A), an input device 213, a compliance module 218, and/or a validation module 219 (“Val. Module 219” in FIG. 2A). The storage 206A of the ear-mountable device 103 may include one or more of raw data 216 and/or detected behaviors/biometrics/conditions (hereinafter “detected parameters”) 220.

The sensor 208 may include one or more of a microphone, an accelerometer, a gyro sensor, a PPG sensor, an ECG sensor, an EDA sensor, a vibration sensor, a light sensor, a UV light sensor, a body temperature sensor, an environmental temperature sensor, or other suitable sensor. While only a single sensor 208 is illustrated in FIG. 2A, more generally the ear-mountable device 103 may include one or more sensors.

In some embodiments, the ear-mountable device 103 may include multiple sensors 208, with a trigger from one sensor 208 causing another sensor 208 to receive power and start capturing data. For example, an accelerometer, gyro sensor, ECG sensor, or other relatively low-power sensor may trigger a microphone to begin receiving power to capture audio data.

The output device 209 may include a speaker or other device to output audio signals to a subject or user. For example, when the ear-mountable device 103 is implemented as a hearing aid, the output device 209 may include a speaker to output sound representative of sound in an environment of the user that has been amplified and/or processed to, e.g., improve speech intelligibility and/or reduce noise. Alternatively or additionally, when the ear-mountable device 103 is implemented as a headphone, the output device 209 may include a speaker to output sound from, e.g., a portable music player, a radio, a computer, or other signal source. In some embodiments, the output device 209 may also be used to output messages, such as compliance messages, queries to provide annotations, or other messages, to the subject.

The input device 213 may include a microphone, accelerometer, or other device to receive input from a subject or user. For example, the user, in response to a query received via the output device 209, may respond to the query by speaking a response aloud, tapping the ear-mountable device 103 with a predetermined number and/or pattern of taps, or providing other input suitable for a given implementation of the input device 213. Although the input device 213 is illustrated as being separate from the sensor 208, alternatively a given one of the sensors 208 may also function as the input device 213.

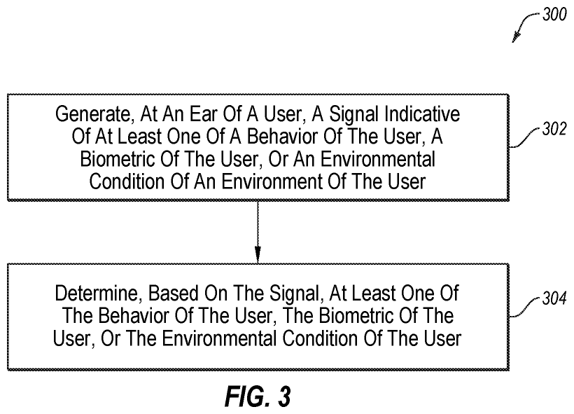

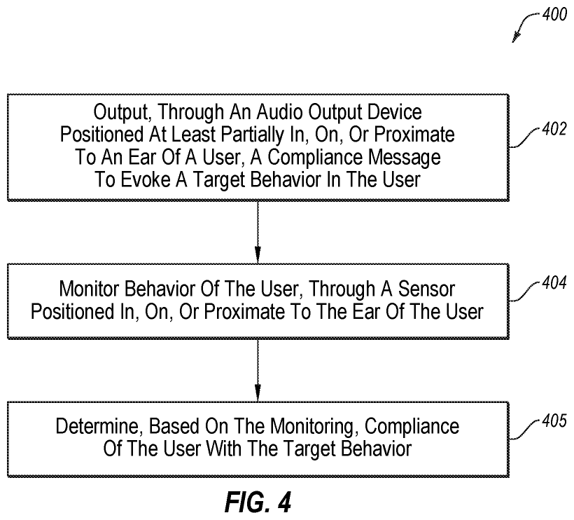

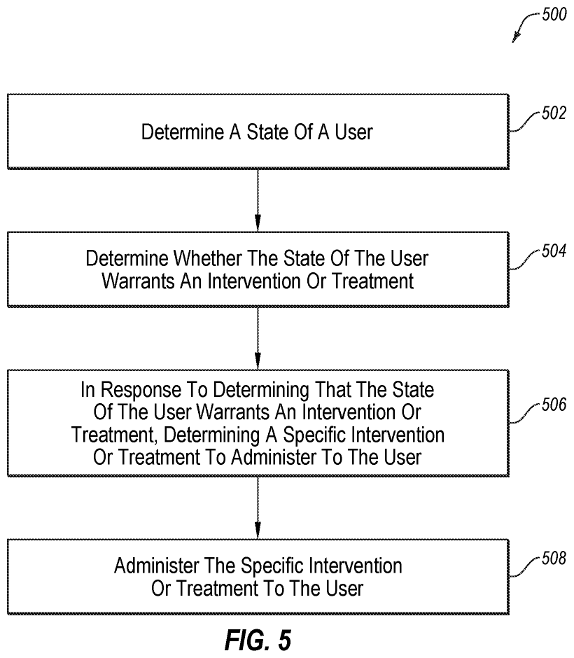

One or more of the intervention module 211, the compliance module 218, and the validation module 219 may each include code such as computer-readable instructions that may be executable by a processor, such as the processor 202A of the ear-mountable device 103 and/or the processor 202B of the remote server 110, to perform or control performance of one or more methods or operations as described herein. For instance, the intervention module 211 may include code executable to perform or control performance of the method and/or one or more of the operations described with respect to FIG. 5. Analogously, the compliance module 218 may include code executable to perform or control performance of the method and/or one or more of the operations described with respect to FIG. 4. Analogously, the validation module 219 may include code executable to perform or control performance of the method and/or one or more of the operations described with respect to FIG. 3.

The raw data 216 may include some or all of each data signal generated by each sensor 208. In an example embodiment, portions of each data signal may be stored temporarily in the storage 206A for processing (e.g., feature extraction as described in the '242 application) and may be discarded after processing, to be replaced by another newly collected portion of the data signal. Alternatively or additionally, one or more portions of one or more data signals may be retained in storage 206A even after being processed. In some embodiments, certain sensors may continuously gather data, while others may intermittently capture data. For example the data 216 may contain continuous data from an accelerometer but only a few windows of data from a microphone.

In some embodiments, the size of the data 216 stored may be based on the capacity of the storage 206A. For example, if the storage 206A includes large amounts of storage, longer windows of time of the data 216 may be stored, while if the storage 206A includes limited amounts of storage, shorter windows of time of the data 216 may be stored. As another example, if the storage 206A includes large amounts of storage, multiple short windows of time of the data 216 may be stored, while if the storage 206A includes limited amounts of storage, a single window of time of the data 216 may be stored.

The detected parameters 220 may include behaviors, biometrics, and/or environmental conditions determined from the signals generated by the sensors 208. Each of the detected parameters 220 may include, e.g., a classification of the parameter, a time at which the parameter occurred, and/or other information.

In some embodiments, the sensors 208 may include a microphone (and/or the input device 213 may include a microphone) and at least one other sensor. The processor 202A may continually monitor the raw data 216 from the other sensor other than the microphone (e.g., an accelerometer). The data 216 from the other sensor may be continuously gathered and discarded along a running window (e.g., storing a window of 10 seconds, discarding the oldest time sample as a new one is obtained). In these and other embodiments, as the raw data 216 for the other sensor is monitored to identify a feature for waking up the microphone (e.g., a rapid acceleration potentially identified as a sneeze), the raw data 216 may include a window of audio data from the microphone. The processor 202A may analyze both the raw data 216 from the other sensor and the raw data 216 from the microphone to extract one or more features 218.

Referring to the remote server 110, it may additionally include a feature extractor 210B, a classifier 212B, and/or a machine learning (ML) module 222. The storage 206B of the remote server 110 may include one or more of subject data 224 and/or detection algorithms 226. The subject data 224 may include snippets of data, extracted features, detected parameters (e.g., behaviors, biometrics, environmental conditions), and/or annotations received from ear-mountable devices, wearable electronic devices, smartphones, and/or sensor devices used by subjects, such as the ear-mountable device 103, the wearable electronic device 104, the smartphone 106, and/or the sensor devices 116 of FIG. 1. The detection algorithms 226 may include algorithms and/or state machines used by the ear-mountable device 103 and/or the remote server 110 in the detection of, e.g., behaviors, biometrics, and/or environmental conditions.

The feature extractor 210B, the classifier 212B, and the ML module 222 may each include code such as computer-readable instructions that may be executable by a processor, such as the processor 202B of the remote server 110, to perform or control performance of one or more methods or operations as described herein. For instance, the feature extractor 210B and the classifier 212B may in some embodiments perform processing of snippets of data signals, extracted features, and/or other data received from the ear-mountable device 103. The ML module 222 may evaluate some or all of the subject data 224 to generate and/or update the detection algorithms 226. For instance, annotations together with extracted features, detected behaviors, detected biometrics, and/or detected environmental conditions or other subject data 224 may be used as training data by the ML module 222 to generate and/or update the detection algorithms 226. Updated detection algorithms 226 used in feature extraction, classification, or other aspects of behavior, biometric, and/or environmental condition detection may then update one or more of the feature extractors 210A, 210B and/or classifiers 212A, 212B or other modules in one or both of the remote server 110 and ear-mountable device 103.

FIGS. 2B and 2C illustrate two ear-mountable devices implemented as hearing aids 250A, 250B (collectively “hearing aids 250”, generically “hearing aid 250”), arranged in accordance with at least one embodiment described herein. FIG. 2B illustrates the hearing aid 250A by itself and FIG. 2C illustrates the hearing aid 250B attached to a user's ear 252.

As illustrated in FIGS. 2B and 2C, each hearing aid 250 includes an ear canal insertion portion 254A, 254B (collectively “ear canal insertion portions 254”, generically “ear canal insertion portion 254”), a main body 256A, 256B (collectively “main bodies 256”, generically “main body 256”), and an ear hook 258A, 258B (collectively “ear hooks 258”, generically “ear hook 258”) between each ear canal insertion portion 254 and corresponding main body 256. As illustrated in FIG. 2C, the ear canal insertion portion 254 may be positioned at least partially within the user's ear-hole 260 and/or the user's ear canal, while the main body 256 may be positioned behind the user's ear 252. The ear hook 258 extends from the ear canal insertion portion 254 over the top of the ear 252 to the main body behind the ear 252 to attach the hearing aid 250 to the user's ear 252.

In general, the main body 256 may include a microphone to convert a voice signal into an electrical signal, a hearing aid processing circuit to amplify the output signal of the microphone and/or perform other such hearing aid processing, an earphone circuit to convert the output of the hearing aid processing circuit into a voice signal, a battery to power the hearing aid 250, and/or other circuits, components, or portions. The ear canal insertion portion 254 may include a speaker to convert the voice signal into sound. The ear hook 258 may provide a mechanical connection and/or an electrical connection between the main body 256 and the ear canal insertion portion 254. The microphone of the hearing aid 250 may include or correspond to the sensor 208 and/or the input device 213 of FIG. 2A. The earphone circuit and/or speaker may include or correspond to the output device 209 of FIG. 2A.

Alternatively or additionally, the hearing aid 250 may include one or more other sensors, such as one or more of a temperature sensor, a PPG sensor, a sweat vapor sensor, a tympanic membrane sensor, an EEG sensor, a UV light sensor, a light sensor, and/or other sensors. The additional sensor(s) may be located in or on the main body 256, the ear hook 258, and/or the ear canal insertion portion 254, depending on the sensor signal that is desired to be acquired. For example, if it is desired to acquire core body temperature, heart rate via PPG, sweat vapor, and/or UV/light levels, the additional sensor may be located in or on the ear canal insertion portion 254 so that the additional sensor is positioned inside the user's ear canal during use. Alternatively or additionally, if it is desired to acquire environmental/ambient temperature/light levels/sound, the additional sensor may be located in or on the main body 256 and/or the ear hook 258 so that the additional sensor is positioned outside the user's ear 252 during use.

Optionally, the main body 256 may be attached behind the user's ear 252, e.g., directly to the skull or directly to the back of the ear 252, using an adhesive to ensure and/or improve conduction of audio waves and/or bone conduction to a sensor included in or on the main body 256.

Optionally, the hearing aid 250 and/or other ear-mountable devices described herein may be communicatively linked to other devices (e.g., the wearable electronic device 104, the smartphone 106, one or more of the sensor devices 116, or other devices). With such a communication link, the hearing aid 250 and/or other ear-mountable devices may receive updates or alerts from the other devices and may output audio updates or alerts to the user. For example, when one of the other devices has a low battery, poor signal quality, or needs to be synchronized to a base station or hub, the other device may send a corresponding update or alert to the hearing aid 250 and/or other ear-mountable device, which may then output an audio update or alert to the user so that the user can take appropriate action.

FIG. 2D illustrates an ear-mountable device implemented as circumaural headphones 262 (hereinafter “headphones 262”), arranged in accordance with at least one embodiment described herein. Other examples of ear-mountable devices, in addition to hearing aids and circumaural headphones, include supra-aural headphones, earbuds, canal phones, and Bluetooth headsets.

As illustrated in FIG. 2D, the headphones 262 include first and second headphone units 264A, 264B (collectively “headphone units 264”) connected by a headband 266. The headphones 262 may additionally include a communication interface, such as a wired or wireless interface, to receive electrical signals representative of sound, such as music. The headphones 262 may additionally include a speaker, such as one or more speakers in each of the headphone units 264, to convert the electrical signals to sound. The speaker(s) may include or correspond to the output device 209 of FIG. 2A.

The headphones 262 may additionally include one or more input devices, such as the input device 213 of FIG. 2A. For example, one or both of the headphone units 264 may include a microphone and/or the microphone may extend downward and forward (e.g., toward a user's mouth when the headphones 262 are in use) from one of the headphone units 264.

Alternatively or additionally, the headphones 262 may include one or more other sensors, such as one or more of a temperature sensor, a PPG sensor, a sweat vapor sensor, a tympanic membrane sensor, an EEG sensor, a UV light sensor, a light sensor, a sound sensor, and/or other sensors. The additional sensor(s) may be located in or on either or both of the headphone units 264 or the headband 266, depending on the sensor signal that is desired to be acquired. For example, if it is desired to acquire EEG waves, the sensor may be located in or on the headband 266.

FIG. 3 is a flowchart of an example validation method 300, arranged in accordance with at least one embodiment described herein. The method 300 may be implemented, in whole or in part, by one or more of the ear-mountable devices 103, the wearable electronic device 104, the smartphone 106, one or more of the sensor devices 116, and/or the remote server 110. Alternatively or additionally, execution of the validation module 219 by the processor 202A and/or 202B of the ear-mountable device 103 and/or the remote server 110 of FIG. 2A may cause the corresponding processor 202A and/or 202B to perform or control performance of one or more of the operations or blocks of the method 300. The method 300 may include one or more of blocks 302 and/or 304. The method 300 may begin at block 302.

At block 302, a signal indicative of at least one of a behavior of a user, a biometric of a user, or an environmental condition of an environment of the user may be generated at an ear of the user. For example, such a signal may be generated by the ear-mountable device 103 of FIG. 2A (or either or both of the ear-mountable devices 103 of FIG. 1), and more particularly by one or more of the sensors 208 of FIG. 2A. The ear-mountable device 103 may be mounted to the user—e.g., the subject 102 of FIG. 1—in, on, or proximate to the ear of the user.

Generating the signal at block 302 may include generating, at the ear of the user, at least one of an audio signal, a bone conduction signal, a vibrational sound signal, an accelerometer signal, a sweat vapor (or component thereof) signal, a light signal, a UV light signal, or a temperature signal. In this and other embodiments, the signal may specifically be indicative of at least one of: the user swallowing; the user grinding the user's teeth; the user chewing; the user coughing; the user vomiting; the user wheezing; the user sneezing; an intoxication state of the user; a dizziness level of the user; the user's heart rate; the user's EEG brain waves; the user's body temperature; the user's sweat vapor to sense volatile organic compounds to determine if the user has consumed a particular substance such as alcohol, ethanol, a medication, or other substance emitted through sweat; an ambient temperature in the environment of the user; an ambient light level in the environment of the user; an ambient UV light level in the environment of the user; ambient music, which may then be analyzed to determine artist, song, genre, or other information to correlate with mood/depression of the user.

Additional details regarding the detection of markers, e.g., of alcohol, medications, or other substances, in sweat vapor are disclosed in co-pending U.S. application Ser. No. 15/353,738 (hereinafter the '738 application) filed Nov. 17, 2016 which is incorporated herein by reference.

Block 302 may be followed by block 304. At block 304, at least one of the behavior of the user, the biometric of the user, or the environmental condition of the environment of the user may be determined based on the signal. In some embodiments, determining at least one of the behavior of the user, the biometric of the user, or the environmental condition of the environment of the user may be determined exclusively based on the signal, e.g., based on a single signal. In other embodiments, determining at least one of the behavior of the user, the biometric of the user, or the environmental condition of the environment of the user may be determined based on two more signals, e.g., generated by two or more sensors.

The method 300 of FIG. 3 may include passive validation or active validation. Passive validation may involve sensing and determining the at least one of the behavior of the user, the biometric of the user, or the environmental condition of the environment of the user passively, e.g., without requesting or receiving any input or action from the user. On the other hand, active validation may involve sensing and determining the at least one of the behavior of the user, the biometric of the user, or the environmental condition of the environment of the user actively, e.g., by requesting and receiving an input from the user, where the input may generally confirm or deny the at least one of the behavior of the user, the biometric of the user, or the environmental condition of the environment of the user.

In an example active validation implementation, the method 300 may further include making a preliminary determination of at least one of the behavior of the user, the biometric of the user, or the environmental condition of the environment of the user, e.g., based on the signal. The method 300 may also include outputting, through an audio output device positioned at least partially in, on, or proximate to the ear of the user, a query regarding the behavior of the user, the biometric of the user, or the environmental condition of the environment of the user. For example, outputting the query may include outputting a query regarding at least one of whether the user performed or exhibited a particular behavior, whether the user is or has been subject to a particular environmental condition, or whether the user is or has been experiencing a particular symptom associated with a particular biometric reading. Various example queries may ask the user whether the user chewed food, swallowed water and/or a medication, ground the user's teeth, vomited, sneezed, coughed, is intoxicated, is dizzy, is nauseous, is or has been subject to a particular environmental condition (e.g., inside a dark room) for at least a predetermined amount of time, and/or is or has been wheezing or has shortness of breath (e.g., which may occur if the user's heartbeat or breathing is racing without any indication that the user is exercising).

In some embodiments, the audio output device may include the output device 209 of FIG. 2A, which may be positioned in, on, or proximate to the ear of the user when mounted to the user. The query may ask or instruct the user to confirm that the preliminarily determined behavior, biometric, or environmental condition actually occurred, e.g., by providing a first predetermined input. For example, the query may instruct the user to say “yes” aloud or tap one of the ear-mountable devices 103, the wearable electronic device 104, the smartphone 106, or one of the sensor devices 116 once (or other predetermined number of times and/or pattern) to confirm that the preliminarily determined behavior, biometric, or environmental condition actually occurred. Alternatively or additionally, the query may at least implicitly ask or instruct the user to provide a different second predetermined input to indicate that the preliminarily determined behavior, biometric, or environmental condition did not occur. For example, the query may instruct the user to say “no” aloud or tap one of the ear-mountable devices 103, the wearable electronic device 104, the smartphone 106, or one of the sensor devices 116 twice (or other predetermined number of times and/or pattern) to indicate that the preliminarily determined behavior, biometric, or environmental condition did not occur. In these and other embodiments, determining the at least one of the behavior of the user, the biometric of the user, or the environmental condition of the environment of the user at block 304 may be based on both the sensor signal generated at block 302 and the response to the query.

The response to the query may be received through an input device, such as the input device 213 of FIG. 2A. When the user is asked or instructed to respond to the query by speaking aloud a response to the query, the input device 213 may include a microphone or other audio input device. When the user is asked or instructed to respond to the query by providing one or more taps on one of the ear-mountable devices 103, the wearable electronic device 104, the smartphone 106, or one of the sensor devices 116, the input device 213 may include an accelerometer or other motion detecting device.

In some embodiments, determining the at least one of the behavior of the user, the biometric of the user, or the environmental condition of the environment of the user may include determining that the behavior of the user is not compliant with a target behavior of the user. In this and other embodiments, the method may further include outputting, through the audio output device which is positioned at least partially in, on, or proximate to the ear of the user, a compliance message to evoke the target behavior in the user. For example, the user may have a prescribed medication and the ear-mountable device may monitor the user to determine whether the user takes the prescribed medication according to a prescribed schedule (e.g., one or more times daily). In response to determining that the user has not complied with the prescribed schedule, one or both of the ear-mountable devices 103 may output a message, e.g., through a corresponding output device 209, to take the prescribed medication. Various example behaviors that may be monitored for compliance may include medication adherence, physical exercise, and physical rehabilitation.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

FIG. 4 is a flowchart of an example compliance method 400, arranged in accordance with at least one embodiment described herein. The method 400 may be implemented, in whole or in part, by one or more of the ear-mountable devices 103, the wearable electronic device 104, the smartphone 106, one or more of the sensor devices 116, and/or the remote server 110. Alternatively or additionally, execution of the compliance module 218 by the processor 202A and/or 202B of the ear-mountable device 103 and/or the remote server 110 of FIG. 2A may cause the corresponding processor 202A and/or 202B to perform or control performance of one or more of the operations or blocks of the method 400. The method 400 may include one or more of blocks 402, 404, and/or 406. The method 400 may begin at block 402.

At block 402, a compliance message to evoke a target behavior in a user may be output through an audio output device positioned at least partially in, on, or proximate to the user's ear. In general, the compliance message may ask or instruct the user to perform a particular behavior, such as taking or applying a medication, performing one or more exercises, performing one or more physical rehabilitation exercises, or following some other protocol. As a specific example, a compliance message may ask or instruct the user to take a first dose (or only dose) of a prescribed medication, e.g., at or by a specified time each day, or may ask or instruct the user to do one or more physical rehabilitation exercises, e.g., at or by a specified time each day. Block 402 may be followed by block 404.

At block 404, behavior of the user may be monitored through a sensor positioned in, on, or proximate to the ear of the user. Monitoring the behavior of the user may include generating one or more sensor signals indicative of the behavior of the user, e.g., as described elsewhere herein, including in connection with block 302 of FIG. 3. For example, generating the one or more sensor signals may include generating, at the ear of the user, at least one of an audio signal, a bone conduction signal, a vibrational sound signal, or an accelerometer signal. Alternatively or additionally, generating the signal indicative of the behavior of the user may include generating the at least one of the audio signal, the bone conduction signal, the vibrational sound signal, or the accelerometer signal indicative of at least one of the user swallowing or otherwise consuming a prescribed medication. Block 404 may be followed by block 406.

At block 406, compliance of the user with the target behavior may be determined based on the monitoring. For example, determining compliance of the user with the target behavior based on the monitoring may include comparing one or more features of the signal indicative of the behavior of the user to one or more target features of a signal indicative of the target behavior and determining that the user's behavior includes the target behavior if the one or more features of the signal indicative of the behavior of the user match the one or more target features of the signal indicative of the target behavior.

Alternatively or additionally, determining compliance of the user with the target behavior based on the monitoring may include determining that the user does not comply with the target behavior within a predetermined period of time from the outputting of the compliance message, or within a predetermined period of time specified in the compliance message. For example, it may be determined that the user does not comply with the target behavior within 30 minutes or some other period of time after the compliance message is output to the user, or within 30 minutes of a time specified in the compliance message. In this and other embodiments, the method 400 may further include outputting a reminder compliance message through the audio output device positioned at least partially in, on, or proximate to the ear of the user. The reminder compliance message may remind the user to perform the particular behavior originally specified in the initial compliance message.

Alternatively or additionally, the method 400 may be combined with one or more steps or operations of one or more of the other methods described herein, such as the method 300 of FIG. 3. For example, the method 400 may further include outputting, through the audio output device positioned at least partially in, on, or proximate to the ear of the user, a compliance query regarding the behavior of the user and whether it complies with the target behavior. In this and other embodiments, the compliance determination at block 406 may be based on both the monitoring of the behavior of the user at block 404 and a response from the user to the compliance query.

FIG. 5 is a flowchart of an example intervention method 500, arranged in accordance with at least one embodiment described herein. The method 500 may be implemented, in whole or in part, by one or more of the ear-mountable devices 103, the wearable electronic device 104, the smartphone 106, one or more of the sensor devices 116, and/or the remote server 110. Alternatively or additionally, execution of the intervention module 211 by the processor 202A and/or 202B of the ear-mountable device 103 and/or the remote server 110 of FIG. 2A may cause the corresponding processor 202A and/or 202B to perform or control performance of one or more of the operations or blocks of the method 500. The method 500 may include one or more of blocks 502, 504, 506, and/or 508. The method 500 may begin at block 502.

At block 502, a state of a user may be determined. The state of the user may be determined from one or more sensor signals generated by one or more sensors included in, e.g., one or both of the ear-mountable devices and/or one or more of the other devices of FIG. 1. The determined state may include a mental and/or emotional state (e.g., depressed, sad, lonely, happy, excited) and/or a physical state (e.g., normal or baseline physical state, tired, fallen down, head impact, sore joint(s) or muscle(s)). Block 502 may be followed by block 504.

At block 504, it may be determined whether the state of the user warrants an intervention or treatment. Some mental and/or physical states may not warrant any intervention or treatment (e.g., happy, excited, normal or baseline, tired), while other mental and/or physical states may warrant an intervention (e.g., depressed, fallen down, head impact). Guidelines for determining whether a state warrants an intervention or treatment may be based on guidelines for a general population and/or may be customized based on the specific user. For example, a young, healthy user in a fallen state, e.g., from a slip and fall on an icy walkway in winter, who relatively quickly stands back up and does not remain in the fallen state for very long may not warrant an intervention or treatment, whereas an older user with arthritis in a fallen state, e.g., due to a loss of balance while walking in the user's own home, who remains in the fallen state for more than a predetermined period of time may warrant an intervention or treatment. Block 504 may be followed by block 506.

At block 506, and in response to determining that the state of the user warrants an intervention or treatment, a specific intervention or treatment to administer to the user may be determined. The specific intervention or treatment to administer may depend on the specific state of the user. Block 506 may be followed by block 508.

At block 508, the specific intervention or treatment may be administered to the user. According to the method 500 of FIG. 5, at least one of the following may hold: the state of the user may be determined based on a signal generated by a sensor device positioned in, on, or proximate to the user's ear; or the specific intervention or treatment may be administered at least in part by an output device positioned in, on, or proximate to the user's ear.

In some embodiments, administering the specific intervention or treatment to the user at block 508 may include at least one of: administering a somatosensory evoked potential (SSEP) evaluation of the user; contacting an emergency response service to notify the emergency response service that the user is in need of assistance; administering a treatment to the user to alter at least one of EEG brain waves, a heart rate, or a breathing rate or pattern of the user; administering neuro-stimulation to an ear canal or ear lobe of the user; or applying a magnetic field to at least a portion of the user's body.

A specific example implementation of the method 500 may include determining at block 502 that a user has fallen and/or the user's head has impacted or been impacted by an object based on a signal generated by a sensor positioned in, on, or proximate to the user's ear. A message may be output to the user, e.g., through the output device 209 positioned in, on, or proximate to the user's ear to ask if the user is okay. If the user answers in the negative and/or doesn't answer at all, e.g., within a predetermined period of time, it may be determined at block 504 that the state of the user warrants an intervention or treatment. At block 506, it may be determined to contact an emergency response service to provide assistance to the user as the specific intervention or treatment to administer to the user. At block 508, the emergency response service may be contacted and informed that the user is in need of assistance. Alternatively or additionally, the ear-mountable device may generate, at the ear of the user, a signal indicative of a biometric of the user, such as the user's heart rate, temperature respiration rate, blood pressure, or other vital sign(s). The user's biometric(s) may be provided to the emergency response service, e.g., in advance of the emergency response service reaching the user. Alternatively or additionally, if the state determined at block 502 includes an impact to the user's head, the emergency response service may be informed, e.g., in advance of reaching the user, that the user may have head trauma.

The methods 300, 400, 500 and/or one or more discrete steps or operations thereof may be combined in any combination.

Alternatively or additionally, embodiments described herein may include a hub or smartphone (such as the smartphone 106 of FIG. 1) in a user's bedroom that senses light exposure (e.g., light levels) while the user is asleep. The proximity of the hub or smartphone to the user may be validated, e.g., by proximity detection of another device (such as any one of sensor devices 116) that is attached to the user, optionally combined with one or more signals from the other device that may biometrically authenticate the user as such. One or more ear-mounted devices (such as the devices 103) or other devices (such as the wearable electronic device 104, the smartphone 106, and/or the sensing devices 116) may provide additional light measurements throughout the day. Optionally, the combination of devices may provide around the clock measurements of light exposure, e.g., periodic measurements such as every 15 minutes or every 60 minutes, 24 hours per day. One or more of the devices may also generate signals relating to the user's activity, sleep, ECG, heart rate, heart rate variability, music (or lack thereof), ambient sound (or lack thereof). The combination of around the clock light measurements and one or more other signals may provide insights into the user's mental health. For example, if the user is sleeping significantly longer than usual and remaining in the dark even during the daytime, it may be determined that the user is depressed. If the user has been prescribed one or more medications to treat depression, embodiments described herein may alternatively or additionally validate whether the user is taking the medications, help the user to comply with taking the medication, and/or facilitate an intervention.

Alternatively or additionally, environmental/ambient sound and/or environmental/ambient music may be monitored and/or sensed by ear-mounted devices and/or other devices described herein in connection with the user's mental health. The sound and/or music may be broken down, e.g., by type, as done for, e.g., the music genome project. Embodiments described herein may more generally form correlations and/or causal links between music, behavior, and environment to objectively monitor and diagnose depression and general anxiety disorder.

Embodiments of the ear-mountable device or devices described herein may include, implement, or provide one or more of the following features and/or advantages:

Unique aspects of a hearing aid or other ear-mountable device mounted to the ear area of a user:

1→It's situated on the head

2→It is generally uncovered

3→The ear canal has solid vibration and sound conduction through skull

4→Brain waves and EEG

One or more of the following may have unique benefits from being sensed in the user's ear:

Core Body Temperature

Ambient Light & UV exposure (Unless wearing a hat, but even then, can sense outdoors) (wrist, chest is often covered) . . . light is not

Ambient temperature→Also rarely covered up

Head Orientation

Impact on the Head (Falling and hitting head, any head impact)

Coughing Sneezing, Vomiting . . . gives unique head motions to those actions

Sensing one or more signals at the ear may accomplish validation, compliance, and/or intervention better than other locations of the body:

Passive Validation:

Behaviors: Chewing Food, Swallowing water or taking a pill, Grinding Teeth (Depression/anxiety), Intoxication of alcohol or substances (head wobbles much more while drunk.

Dizziness, Vertigo . . .

Some embodiment s may break down the sound and music the user listens to for correlating mental health independent of knowing what song/album/artist is actually playing. This can correlate with mood/depression, and other states.

Environment: Ambient temperature and light sensing at an ear-mountable device is much better than on wrist/chest which is often covered by clothing.

Biometrics: HR, Coughing/Vomiting/Wheezing, EEG brain waves to assess mood, stress, etc.

Active Validation:

May include having the user use their voice to journal inputs or acknowledge things (e.g., I took medicine, I feel better today, my knee hurts, I have phlegm in my cough).

May include having the ear-mountable device prompt and then user can use voice, tap a sticker sensor several times, or use a smartwatch/smartphone touch screen to respond.

Compliance:

May include hearing aids, headphones, or other ear-mountable devices for medication reminders, rehab reminders for exercise or instructions to follow a protocol. Embodiments herein may measure whether compliance occurs for a user, and then if it is determined that compliance has not occurred, some embodiments may remind the user again. For example, if swallowing or drinking water (e.g., to take a medication) is not detected, some embodiments may remind the user again to take the medication or ask the user for an explicit confirmation that the user took the medication.

Interventions & Treatment:

Some embodiments may involve SSEP, e.g., as described at: http ://ormonitoring.com/what-is-ssep-somatosensory-evoked-potentials/. SSEP may evaluate nerve pathways responsible for feeling touch and pressure. When you touch something hot or step on something sharp, a signal is sent to your brain to react. SSEPs evaluate this signal as it travels to your brain and provide information about the various functions that are important to your sensory system. Understanding sensory function during surgery plays a critical role in detecting and avoiding unintended complications that could leave a patient with short or long term impairment.

SSEP testing involves the stimulation of specific nerves and the recording of their activity as they travel to the brain. Stimulating electrodes are placed over specific nerves, typically at the ankle and/or wrist, while recording electrodes are placed on the scalp over the sensory area of the brain. Function of the sensory pathway is evaluated by measuring the commute time between the nerve and the brain, as well as the strength of the sensory response. If the commute time is slower than expected or if the sensory response is weak, this may indicate abnormalities that are interfering with the pathway.

SSEPs are useful for a variety of reasons, from the evaluation of spinal cord integrity after injury to the assessment of vascular flow to the brain. Due to its ease of application and multi-functional use, SSEPs are often combined with other intraoperative neurophysiologic tests that focus on motor or movement function, such as Electromyography (EMG) or Transcranial Motor Evoked Potentials (TceMEP). SSEP testing is standard practice for intraoperative neuromonitoring during cervical, thoracic, vascular, and brain surgeries, among others.

The SSEP test is a non-invasive way to assess the somatosensory system. While there is always a small risk of infection any time a needle is involved, risks are almost nonexistent otherwise.

Accordingly, some embodiments described herein may send an electrical signal from an ear-mountable device into ear or skull and measure the value at the base of the spine or other location with, e.g., a sticker sensor.

Some embodiments may involve personal emergency response: Example embodiments may detect a fall, and potentially a head impact. The user may be asked if they are ok through the ear-mountable device, and an emergency response service may be called and dispatchers may be informed that there may be head trauma. Alternatively or additionally, vitals may be determined, e.g., from the ear-mountable device or other devices, and may be given to the dispatchers ahead of time before emergency response service personnel arrive.

Some embodiments may involve EEG, breathing, heart rate, with music, activity, etc.

Some embodiments may send neurostimulation to the ear canal or ear lobes for mental priming.

Some embodiments may induce magnetism to treatments.

The present disclosure is not to be limited in terms of the particular embodiments described herein, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that the present disclosure is not limited to particular methods, reagents, compounds, compositions, or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

What is claimed is:

1. A validation method, comprising:
generating, at an ear of a user, a signal indicative of at least one of a behavior of the user, a biometric of the user, or an environmental condition of an environment of the user; and
determining, based on the signal, at least one of the behavior of the user, the biometric of the user, or the environmental condition of the environment of the user.

2. The method of claim 1, wherein the determining is based exclusively on the signal.

3. The method of claim 1, wherein the generating comprises generating, at the ear of the user, at least one of an audio signal, a bone conduction signal, a vibrational sound signal, an accelerometer signal, a temperature signal, a light signal, or an ultraviolet (UV) light signal.

4. The method of claim 3, wherein the generating the signal indicative of at least one of the behavior of the user, the biometric of the user, or the environmental condition of the environment of the user comprises generating the at least one of the audio signal, the bone conduction signal, the vibrational sound signal, the accelerometer signal, the temperature signal, the light signal, or the UV light signal indicative of at least one of the user swallowing; the user grinding the user's teeth; the user chewing; the user coughing; the user vomiting; the user wheezing; the user sneezing; an intoxication state of the user; a dizziness level of the user; the user's heart rate; the user's Electroencephalography (EEG) brain waves; the user's body temperature; the user's blood pressure, the user's breathing rate, the user's sweat vapor to sense volatile organic compounds to determine if the user has consumed a particular substance such as alcohol, ethanol, a medication, or other substance emitted through sweat; an ambient temperature in the environment of the user; an ambient light level in the environment of the user, an ambient ultraviolet (UV) light level in the environment of the user, or ambient music in the environment of the user.

5. The method of claim 1, further comprising determining a mental health state of the user based on ambient light level in the environment of the user and at least one of physical activity level, sleep, ECG brain waves, heart rate, heart rate variability, and ambient music in the environment of the user.

6. The method of claim 1, further comprising:
communicatively coupling an ear-mountable device that generates the signal to at least one of a medical device, a sensor sticker, a wearable electronic device, or a smartphone;
receiving at the ear-mountable device an update or alert from the at least one of the medical device, the sensor sticker, the wearable electronic device, or the smartphone; and
outputting an audio update or alert to the user through an audio output device of the ear-mountable device to inform the use of a low battery, poor signal quality, a synchronization requirement, or other condition affecting the at least one of the medical device, the sensor sticker, the wearable electronic device, or the smartphone.

7. The method of claim 1, further comprising:
making a preliminary determination of at least one of the behavior of the user, the biometric of the user, or the environmental condition of the environment of the user; and
outputting, through an audio output device positioned at least partially in, on, or proximate to the ear of the user, a query regarding the behavior of the user, the biometric of the user, or the environmental condition of the environment of the user;
wherein the determining is based on both the signal and a response to the query.

8. The method of claim 7, further comprising receiving the response to the query at an input device positioned in, on, or proximate to the ear of the user.

9. The method of claim 8, wherein the receiving comprises receiving the response to the query at a microphone positioned in, on, or proximate to the ear of the user.

10. The method of claim 7, further comprising receiving the response to the query at an input device positioned remote from the ear of the user.

11. The method of claim 10, wherein the receiving comprises receiving the response to the query at a wearable device that includes an accelerometer, the wearable device positioned on the user at a location remote from the ear of the user.

12. The method of claim 7, wherein the outputting the query comprises outputting a query regarding at least one of whether the user performed or exhibited a particular behavior, whether the user is or has been subject to a particular environmental condition, or whether the user is or has been experiencing a particular symptom associated with a particular biometric reading.

13. The method of claim 1, wherein the determining comprises determining that the behavior of the user is not compliant with a target behavior of the user, the method further comprising outputting, through an audio output device positioned at least partially in, on, or proximate to the ear of the user, a compliance message to evoke the target behavior in the user.

14. A compliance method, comprising:
outputting, through an audio output device positioned at least partially in, on, or proximate to an ear of a user, a compliance message to evoke a target behavior in the user;
monitoring behavior of the user, through a sensor positioned in, on, or proximate to the ear of the user; and
determining, based on the monitoring, compliance of the user with the target behavior.

15. The method of claim 14, wherein the determining comprises determining that the user does not comply with the target behavior within a predetermined period of time from the outputting of the compliance message, the method further comprising outputting a reminder compliance message through the audio output device positioned at least partially in, on, or proximate to the ear of the user.

16. The method of claim 14, wherein the monitoring comprises generating a signal indicative of the behavior of the user.

17. The method of claim 16, wherein the generating comprises generating, at the ear of the user, at least one of an audio signal, a bone conduction signal, a vibrational sound signal, or an accelerometer signal.

18. The method of claim 17, wherein the generating the signal indicative of the behavior of the user comprises generating the at least one of the audio signal, the bone conduction signal, the vibrational sound signal, or the accelerometer signal indicative of at least one of the user swallowing or otherwise consuming a prescribed medication.

19. The method of claim 16, wherein the determining comprises comparing one or more features of the signal indicative of the behavior of the user to one or more target features of a signal indicative of the target behavior and determining that the user's behavior includes the target behavior if the one or more features of the signal indicative of the behavior of the user match the one or more target features of the signal indicative of the target behavior.

20. The method of claim 14, further comprising outputting, through the audio output device positioned at least partially in, on, or proximate to the ear of the user, a compliance query regarding the behavior of the user and whether it complies with the target behavior, wherein the determining is based on both the monitoring and a response to the compliance query.

21. An intervention method, comprising:
determining a state of a user;
determining whether the state of the user warrants an intervention or treatment;
in response to determining that the state of the user warrants an intervention or treatment, determining a specific intervention or treatment to administer to the user; and
administering the specific intervention or treatment to the user;
wherein at least one of:
the state of the user is determined based on a signal generated by a sensor positioned in, on, or proximate to the user's ear; or
the specific intervention or treatment is administered at least in part by an output device positioned in, on, or proximate to the user's ear.

22. The method of claim 21, wherein administering the specific intervention or treatment to the user comprises at least one of:
administering a somatosensory evoked potential (SSEP) evaluation of the user;
contacting an emergency response service to notify the emergency response service that the user is in need of assistance;
administering a treatment to the user to alter at least one of Electroencephalography (EEG) brain waves, a heart rate, or a breathing rate or pattern of the user;
administering neuro-stimulation to an ear canal or ear lobe of the user; or applying a magnetic field to at least a portion of the user's body.

23. The method of claim 21, wherein determining the state of the user comprises determining the state of the user based on a signal generated by a sensor positioned in, on, or proximate to an ear of the user.

24. The method of claim 21, wherein determining the state of the user comprises determining at least one of:
that the user has fallen, or
that the user's head has impacted or been impacted by an object based on a signal generated by a sensor positioned in, on, or proximate to an ear of the user.

25. The method of claim 21, wherein administering the specific intervention or treatment to the user comprises contacting an emergency response service to notify the emergency response service that the user is in need of assistance, the method further comprising:
generating, at an ear of a user, a signal indicative of a biometric of the user, the biometric of the user including at least one of the user's heart rate, respiration, body position, recorded voice signal, location, temperature, or blood pressure; and
communicating the biometric of the user to the emergency response service.
